(19) United States
(12) Patent Application Publication
(10) Pub. No.: US 2024/0225982 A9
(43) Pub. Date: Jul. 11, 2024

(54) ANTIBACTERIAL SYSTEM WITH NATURALLY DERIVED INGREDIENTS AND COMPOSITIONS COMPRISING THEM

(71) Applicant: Conopco, Inc., d/b/a UNILEVER, Englewood Cliffs, NJ (US)

(72) Inventor: Deidre Lee Mitchell, Oxford, CT (US)

(73) Assignee: Conopco, Inc., d/b/a UNILEVER, Englewood Cliffs, NJ (US)

(21) Appl. No.: 18/276,932

(22) PCT Filed: Feb. 2, 2022
(86) PCT No.: PCT/EP2022/052478

§ 371 Date: Aug. 11, 2023

(30) Foreign Application Priority Data
Feb. 16, 2021 (EP) 21157304.3

(51) Int. Cl. A61K 8/49; A61K 8/34; A61K 8/35; A61K 8/368; A61Q 5/02; A61Q 19/10

(52) U.S. Cl. CPC A61K 8/498; A61K 8/347; A61K 8/35; A61K 8/368; A61Q 5/02; A61Q 19/10; A61K 2800/524; A61K 2800/5922; A61K 2800/74

(57) ABSTRACT

Antibacterial systems with naturally derived ingredients and compositions comprising them are described. The systems have a first ingredient comprising thymol and a second natural ingredient that includes p-anisic acid, aloe, gluconolactone, tetrahydrocurcumin, 4-hydroxyacetophenone or a mixture thereof.

### FIELD OF THE INVENTION

The present invention is directed to an antibacterial system comprising naturally derived ingredients. More particularly, the invention relates to superior thymol comprising antibacterial systems that can be included in end use formulations, including cosmetic, personal care and wash compositions. The antibacterial system of the present invention comprises, in addition to thymol, a second component that is naturally derived and that includes p-anisic acid, aloe, gluconolactone, tetrahydrocurcumin, 4-hydroxyacetophenone or a mixture thereof. Such a system unexpectedly delivers superior antibacterial benefits when added to an end use composition.

### BACKGROUND OF THE INVENTION

DMDM hydantoin, parabens, methylisothiazolinone as well as metylchloroisothiazolinone are commonly used preservatives found in consumer products. Such preservatives have been used for years and are known to work well at maintaining the integrity and stability of certain end use compositions. Nevertheless, there is a desire to develop preservative systems that include naturally occurring components suitable to work well across a full range of consumer products. Naturally occurring preservative systems should be effective at preserving products, not be skin sensitizing and not negatively impact the sensorial characteristics of consumer products, especially those that are topically applied.

In addition to delivering superior antibacterial benefits, the preservative systems should not be harmful to the environment and gentle enough for use on the most fragile consumer, babies.

This invention, therefore, is directed to an antibacterial system with naturally occurring ingredients as well as compositions that comprise the same. The preservative system comprises thymol as a first natural component in addition to a second naturally occurring component that includes p-anisic acid, aloe, gluconolactone, tetrahydrocurcumin, 4-hydroxyacetophenone or a mixture thereof. Such a system unexpectedly delivers superior antibacterial benefits to end use compositions they are used in.

### Additional Information

Efforts have been disclosed for making preservative systems. In U.S. Published Patent Application 2012/0190744A1, preservative systems for cosmetic formulations are described.

Still other efforts have been disclosed for making preservative systems. In U.S. Pat. No. 7,754,774B2, antiseptic disinfectants with 1,2-octane diol are described

Even other efforts have been disclosed for making preservative systems. In U.S. Pat. No. 7,582,68162, antimicrobial active compounds with 1,2-alkane diols are described.

None of the additional information above describes an antibacterial system and end use composition with such a system as described and claimed in the present application.

### SUMMARY OF THE INVENTION

In a first aspect, the present invention is directed to an antibacterial system comprising:
(a) a first component comprising thymol; and
(b) a second component comprising p-anisic acid, aloe, gluconolactone, tetrahydrocurcumin, 4-hydroxyacetophenone or a mixture thereof.

In a second aspect, the present invention is directed to an end use composition comprising:
(a) a first component comprising thymol; and
(b) a second component comprising p-anisic acid, aloe, gluconolactone, tetrahydrocurcumin, 4-hydroxyacetophenone or a mixture thereof.

In a third aspect, the present invention is directed to a use or method for preserving a composition with a combination having a first component comprising thymol and a second component comprising p-anisic acid, aloe, gluconolactone, tetrahydrocurcumin, 4-hydroxyacetophenone or a mixture thereof.

All other aspects of the present invention will more readily become apparent from the description and examples which follow.

Antibacterial system, as used herein, means at least a two-component and natural system suitable to show a synergistic antibacterial benefit, including an antibacterial benefit as seen with preservatives. The at least two component system means comprising thymol and a second component comprising p-anisic, aloe, gluconolactone, tetrahydrocurcumin, 4-hydroxyacetophenone or a mixture thereof where the same is an additive for end use compositions (i.e., can be sold as a separate additive composition for adding to an end use composition or formulated separately along with the ingredients to make an end use composition). Naturally derived means not requiring synthetic manufacturing and suitable to be recovered from (at least in precursor form), for example, a plant or root. Skin, as used herein, includes skin on the feet, face, neck, chest, arms (including under arms), hands, legs, buttocks, back and scalp (including hair). The end use composition described herein (i.e., a composition ready to use or apply) includes creams, lotions, serums, gels, balms, deodorants and antiperspirants, oral care compositions, shampoos, conditioners, bars and liquid wash products as well as home care compositions like hard surface and window cleaners, toilet bowl cleaners and laundry detergents. In one embodiment, the end use composition of the present invention is a cleaning composition, a wash product or a leave-on product, such as a cream or lotion to be applied to the face, body or hands. In another embodiment, the end use composition is a cosmetic leave-on product suitable to cosmetically reduce the appearance of wrinkles and/or moisturize skin. Such a composition can also be one that results in skin having and an even colour or tone.

Unless explicitly stated otherwise, all ranges described herein are meant to include all ranges subsumed therein. The term comprises is meant to encompass the terms consisting essentially of and consisting of. For the avoidance of doubt, a composition comprising thymol and p-anisic acid is meant to include a composition consisting essentially of the same and a composition consisting of the same. As to the percentages used herein, the same are meant to be by weight unless noted otherwise.

Except in the operating and comparative examples, or where otherwise explicitly indicated, all numbers used in this description indicating amounts, or ratios of materials and/or use thereof are to be understood as modified by the word “about”.

### DETAILED DESCRIPTION OF THE INVENTION

There is no limitation with respect to how the thymol used in the present invention is sourced except that obtaining of the same is preferably achieved in a most sustainable manner. Often, pressurized liquid extraction or supercritical fluid extraction of thyme can be used to extract thymol. Other techniques include recovering thymol from plants at ambient pressure and at a temperature from 30 to 45° C. when extracting with limonene and ethanol. Alternatively, thyme oil and/or thyme extract comprising thymol may be added to the antibacterial composition of this invention as long as the oil and extract possess thymol. Thyme oil and extract are obtained from the thyme plant. Such a plant belongs to the genus thymus and includes, but is not limited to, the following species: Thymus vulgaris, Thymus zygis, Thymus satureoides, Thymus mastichina, Thymus broussaneti, Thymus maroccamus, Thymus pallidus, Thymus algeriensis, Thymus serpyllum, Thymus pulegoide, and Thymus citrodorus.

Typically, the end use composition having a first component comprising thymol will have from 0.001 to 6% thymol. In an embodiment of the invention, the composition will have from 0.01 to 4%, and still another embodiment, from 0.1 to 3% by weight thymol, based on total weight of the end use composition. To the extent thyme oil and/or extract is used in the end use composition, the same will be added at amounts to ensure that the thymol level in the end use composition is consistent with the levels described herein.

As to the second component comprising p-anisic acid, aloe, gluconolactone, tetrahydrocurcumin, 4-hydroxyacetophenone or a mixture thereof, such components are conventionally recovered from natural sources. P-anisic acid (4-methoxybenzoic acid) is found naturally in anise, and aloe is recovered from the aloe vera plant. Gluconolactone is obtained from, for example, corn. Tetrahydrocurcumin is a metabolite of curcumin (found in turmeric) and 4-hydroxyacetophenone can be found in and recovered from tomato, cassia and cocoa.

The second component of the antibacterial system of the present invention will typically make up from 0.001 to 6% by weight of the end use composition. In another embodiment, from 0.01 to 4.0%, and still another embodiment, from 0.1 to 3.0% by weight of the end use composition, based on total weight of the end use composition.

With respect to the antibacterial system having a first component comprising thymol and second component comprising p-anisic acid, aloe, gluconolactone, tetrahydrocurcumin, 4-hydroxyacetophenone or a mixture thereof, typically the weight ratio of first component to second component in the antibacterial system is from 1:6 to 6:1. In another embodiment, the weight ratio of first component to second component is 4:1 to 1:4, and in still another embodiment, from 2:1 to 1:2, including all ratios subsumed therein. The antibacterial system can consist of thymol and second component, and however, it is within the scope of the invention for the antibacterial system to comprise or consist essentially of thymol and second component. In still another embodiment of the present invention, the antibacterial system of the present invention consists essentially of or consists of thymol and p-anisic acid, aloe, gluconolactone, tetrahydrocurcumin, 4-hydroxyacetophenone or a mixture thereof where no additional antibacterial components are required in the antibacterial system. In yet another embodiment, the antibacterial system can further include fragrance oil and/or a fragrance (from 0.1 to 6% by weight of the antibacterial system) and/or water (from 1 to 25% by weight of the antibacterial system). In such an embodiment, the fragrance and/or fragrance oil is/are provided to deliver a desired scent or aroma to a consumer.

There is generally no limitation with respect to the type of end use composition that may comprise the antibacterial system of the present invention. End use compositions that are suitable to use the antibacterial systems will typically include cosmetically acceptable carrier components. Water is the most preferred carrier. Amounts of water may range from 1 to 96%, and preferably from 5 to 90%, and most preferably, from 35 to 80%, and optimally, from 40 to 75% by weight, based on total weight of the end use composition. Ordinarily the end use compositions of this invention will be water and oil emulsions, most preferably, of the oil-in-water variety. Water-in-oil emulsions, and especially, those generally classified as water-in-oil and high internal phase emulsions are, however, an option. Illustrative examples of the high internal phase emulsions suitable to include the antibacterial systems of this invention are described in commonly owned U.S. Patent Application Publication No. 2008/0311058 and U.S. Pat. No. 8,425,882, the disclosures of which are incorporated herein by reference.

Other cosmetically acceptable carriers suitable for use in this invention may include mineral oils, silicone oils, synthetic or natural esters, and alcohols. Amounts of these materials may range from 0.1 to 50%, and preferably, from 0.1 to 30%, and most preferably, from 1 to 20% by weight of the end use composition, including all ranges subsumed therein. In still another embodiment, such carriers collectively make up from 1 to 12% by weight of the end use composition.

Silicone oils may be divided into the volatile and non-volatile variety. The term “volatile” as used herein refers to those materials which have a measurable vapor pressure at ambient temperature. Volatile silicone oils are preferably chosen from cyclic or linear polydimethylsiloxanes containing from 3 to 9, and preferably, from 4 to 5 silicon atoms.

Linear volatile silicone materials generally have viscosities of less than 5 centistokes at 25° C. while cyclic materials typically have viscosities of less than 10 centistokes (measured with a Brookfield Viscometer, RV No. 3 spindle at 20 RPM, standardized to mineral oil).

Nonvolatile silicone oils useful as carrier material include polyalkyl siloxanes, polyalkylaryl siloxanes and polyether siloxane copolymers. The essentially non-volatile polyalkyl siloxanes useful herein include, for example, polydimethylsiloxanes (like dimethicone) with viscosities of from 5 to 100,000 centistokes at 25° C.

An often-preferred silicone source is a cyclopentasiloxane and dimethiconol solution.

Among suitable esters are:
(1) alkyenyl or alkyl esters of fatty acids having 10 to 20 carbon atoms like isopropyl palmitate, isopropyl isostearate, isononyl isonanonoate, oleyl myristate, isopropyl myristate, oleyl stearate, and oleyl oleate;
(2) ether-esters such as fatty acid esters of ethoxylated fatty alcohols;
(3) polyhydric alcohol esters such as ethylene glycol mono- and di-fatty acid esters, diethylene glycol mono- and di-fatty acid esters, polyethylene glycol (200-6000) mono- and di-fatty acid esters, propylene glycol mono- and di-fatty acid esters, polypropylene glycol 2000 monooleate, polypropylene glycol 2000 monostearate, ethoxylated propylene glycol monostearate, glyceryl mono- and di-fatty acid esters, polyglycerol poly-fatty esters, ethoxylated glyceryl monostearate, 1,3-butylene glycol monostearate, 1,3-butylene glycol distearate, polyoxyethylene polyol fatty acid esters, sorbitan fatty acid esters, and polyoxyethylene sorbitan fatty acid esters;
(4) wax esters such as beeswax, spermaceti, myristyl myristate, stearyl stearate; and (5) sterol esters, of which soya sterol and cholesterol fatty acid esters are examples thereof.

Emulsifiers (or surfactants) may be present in the end use compositions comprising the antibacterial systems of the present invention. Total concentration of the emulsifier may range from 0.1 to 30%, and preferably, from 2 to 20%, and most preferably, from 1 to 8% by weight of the end use composition. The emulsifier may be selected from the group consisting of anionic, nonionic, cationic and amphoteric components. Particularly preferred nonionic components are those with a C₁₀ to C₂₀ fatty alcohol or acid hydrophobe condensed with from 2 to 100 moles of ethylene oxide or propylene oxide per mole of hydrophobe; C₂-C₁₀ alkyl phenols condensed with from 2 to 20 moles of alkylene oxide; mono- and di-fatty acid esters of ethylene glycol; fatty acid monoglyceride; sorbitan, mono- and di-C₈-C₂₀ fatty acids; and polyoxyethylene sorbitan as well as combinations thereof. Alkyl polyglycosides and saccharide fatty amides (e.g. methyl gluconamides) are also suitable nonionic emulsifiers.

Preferred anionic emulsifiers include alkyl ether sulfate and sulfonates, alkyl sulfates and sulfonates, alkylbenzene sulfonates, alkyl and dialkyl sulfosuccinates, C₈-C₂₀ acyl isethionates, C₈-C₂₀ alkyl ether phosphates, alkylethercarboxylates and combinations thereof.

Cationic emulsifiers that may be used include, for example, palmitamidopropyltrimonium chloride, distearyldimonium chloride and mixtures thereof. Useful amphoteric emulsifiers include cocoamidopropyl betaine, C₁₂-C₂₀ trialkyl betaines, sodium lauroamphoacetate, and sodium laurodiamphoacetate or a mixture thereof.

Other generally preferred emulsifiers include glyceryl stearate, glycol stearate, stearamide AMP, PEG-100 stearate, cetyl alcohol as well as emulsifying/thickening additives like hydroxyethylacrylate/sodium acryloyldimethyl taurates copolymer/squalene and mixtures thereof.

While not required, traditional preservatives can optionally be incorporated into the end use compositions comprising the antibacterial systems of this invention to assist against the growth of potentially harmful microorganisms. Suitable traditional preservatives for optional use in the compositions of this invention include alkyl esters of para-hydroxybenzoic acid. Other preservatives include hydantoin derivatives, propionate salts, and a variety of quaternary ammonium compounds. Often preferred preservatives are sodium benzoate, iodopropynyl butyl carbamate, phenoxyethanol, methyl paraben, propyl paraben, imidazolidinyl urea, sodium dehydroacetate and benzyl alcohol. Especially preferred additives suitable to be employed with or without traditional preservatives optionally employed in this invention are 1,2-alkanediols (like 1,2-octanediol and 1,2 hexanediol).

Traditional fragrance components (added separately or with the fragrance in the end use composition) like eugenol, coumarin, linalyl acetate, citronellal, iris concentrate, terpinyl acetate, pinenes (alpha and beta pinene) and citronellol may optionally be added.

If employed, the traditional preservatives, vicinal diol and/or fragrance component will not make up more than 2%, and preferably, not more than 1%, and most preferably, not more than 0.6% by weight of the end use composition of the present invention. In an embodiment of this invention from 0.0001 to 0.85% by weight optional preservative, vicinal diol and/or fragrance component is used, based on total weight of the end use composition. In a preferred embodiment of the invention, no traditional preservative, vicinal diol and/or fragrance component (except for what may be provided in the fragrance used in the end use composition) is used in the end use composition since such compositions comprise the antibacterial system of the present invention.

Thickening agents may optionally be included in end use compositions of the present invention. Particularly useful are the polysaccharides. Examples include citrus fibers, starches, natural/synthetic gums and cellulosics. Representative of the starches are chemically modified starches such as sodium hydroxypropyl starch phosphate and aluminium starch octenylsuccinate. Tapioca starch is often preferred. Suitable gums include xanthan, sclerotium, pectin, karaya, Arabic, agar, guar, carrageenan, alginate and combinations thereof. Suitable cellulosics include hydroxypropyl cellulose, hydroxypropyl methylcellulose, ethylcellulose and sodium carboxy methylcellulose. Synthetic polymers are yet another class of effective thickening agent. This category includes crosslinked polyacrylates such as the Carbomers, polyacrylamides such as Sepigele 305 and taurate copolymers such as Simulgel EGO and Aristoflexe AVC, the copolymers being identified by respective INCI nomenclature as Sodium Acrylate/Sodium Acryloldimethyl Taurate and Acryloyl Dimethyltaurate/Vinyl Pyrrolidone Copolymer. Another preferred synthetic polymer suitable for thickening is an acrylate-based polymer made commercially available by Seppic and sold under the name Simulgel IN100.

Amounts of the thickener, when used, may range from 0.001 to 5%, and preferably, from 0.1 to 2%, and most preferably, from 0.2 to 0.5% by weight of the end use composition.

Fragrances, fixatives and abrasives may optionally be used in the end use compositions that include the antibacterial systems of the present invention. Each of these substances may range from 0.05 to 5%, preferably between 0.1 and 3% by weight.

Conventional humectants may be employed in the compositions of the present invention. These are generally polyhydric alcohol-type materials. Typical polyhydric alcohols include glycerol (i.e., glycerine or glycerin), propylene glycol, dipropylene glycol, polypropylene glycol, polyethylene glycol, sorbitol, hydroxypropyl sorbitol, hexylene glycol, 1,3-butylene glycol, isoprene glycol, 1,2,6-hexanetriol, ethoxylated glycerol, propoxylated glycerol and mixtures thereof. Most preferred is glycerine, propylene glycol or a mixture thereof. The amount of humectant employed may range anywhere from 0.5 to 20%, preferably between 1 and 15%, and most preferably, from 2 to 10% by weight of the end use composition.

The end use compositions of the present invention may include vitamins. Illustrative vitamins are Vitamin A (retinol) as well as retinol esters like retinol palmitate and retinol propionate, Vitamin B2, Vitamin B3 (niacinamide), Vitamin B6, Vitamin C, Vitamin E, Folic Acid and Biotin. Derivatives of the vitamins may also be employed. For instance, Vitamin C derivatives include ascorbyl tetraisopalmitate, magnesium ascorbyl phosphate and ascorbyl glycoside. Derivatives of Vitamin E include tocopheryl acetate, tocopheryl palmitate and tocopheryl linoleate. DL-panthenol and derivatives may also be employed. Total amount of vitamins when present may range from 0.001 to 10%, and preferably from 0.01% to 5%, optimally from 0.1 to 2% by weight of the end use composition.

Other optional additives suitable for use in this invention include resorcinols like 4-ethyl resorcinol, 4-hexyl resorcinol, 4-phenylethyl resorcinol, dimethoxytoluyl propyl resorcinol, 4-cyclopentyl resorcinol, 4-cyclohexylresorcinol, alpha- and/or beta-hydroxyacids, petroselinic acid, conjugated linoleic acid, 12-hydroxystearic acid, mixtures thereof or the like. Still other optional additives like ethanol, quaternary ammonium compounds (like cetrimonium chloride, benzalkonium chloride or the like) and lecithin may also be included. Such additives, when used, collectively make up from 0.001 to 12%, and preferably, from 0.01 to 6%, and most preferably, from 0.1 to 4% by weight of the end use composition.

Desquamation promoters may be present. Illustrative are the alpha-hydroxycarboxylic acids, beta-hydroxycarboxylic acids. The term “acid” is meant to include not only the free acid but also salts and C₁-C₃₀ alkyl or aryl esters thereof and lactones generated from removal of water to form cyclic or linear lactone structures. Representative acids are glycolic and its derivatives, lactic and malic acids. Salicylic acid is representative of the beta-hydroxycarboxylic acids. Amounts of these materials when present may range from 0.01 to 15%, and preferably, from 0.1 to 6% by weight of the end use composition.

A variety of herbal extracts may optionally be included in the end use compositions of this invention. The extracts may either be water soluble or water-insoluble carried in a solvent which respectively is hydrophilic or hydrophobic. Water and ethanol are the preferred extract solvents. Illustrative extracts include those removed from green tea, yarrow, chamomile, licorice, aloe vera, grape seed, citrus unshiu, willow bark, sage and rosemary.

Also optionally suitable for use include materials like chelators (e.g., EDTA), opacifiers (like TiO₂, particle size from 50 to 1200 nm, and preferably, 50 to 350 nm), C₈₋₂₂ fatty acid substituted saccharides, lipoic acid, retinoxytrimethylsilane (available from Clariant Corp. under the Silcare 1M-75 trademark), dehydroepiandrosterone (DHEA) and combinations thereof. Ceramides (including Ceramide 1, Ceramide 3, Ceramide 3B and Ceramide 6) as well as pseudoceramides may also be optionally included. Amounts of these materials may range from 0.0001 to 10%, and preferably, from 0.001 to 6%, and most preferably, from 0.001 to 3% by weight of the end use composition when they are used.

Sunscreen actives may also be optionally included in end use compositions of the present invention. Particularly preferred are such materials as ethylhexyl p-methoxycinnamate, available as Parsol MCX®, Avobenzene, available as Parsol 1789® and benzophenone-3, also known as Oxybenzone. Inorganic sunscreen actives may be employed such as microfine titanium dioxide, zinc oxide, polyethylene and various other polymers. Amounts of the sunscreen agents when present may generally range from 0.1 to 30%, preferably from 0.5 to 20%, optimally from 0.75 to 10% by weight.

Conventional buffers/pH modifiers may be used. These include commonly employed additives like sodium hydroxide, potassium hydroxide, hydrochloric acid, citric acid, triethanolamine, citrate/citric acid buffers or mixtures thereof. In an especially preferred embodiment, the pH of the composition of this invention is from 4 to 8, and preferably, from 4.25 to 7.75, and most preferably, from 5.6 to 7.5.

In an embodiment of the invention and when the end use composition is a conventional cleansing or wash composition, the same often comprises 10 to 45% by weight C₈ to C₂₀, preferably C₁₀ to C₂₀ or C₁₂ to C₁₈ fatty acid or fatty acid soap and less than 5% by weight synthetic surfactant, and preferably, 1 to 3% by weight synthetic surfactant. In an embodiment of the invention, the cleansing or wash composition comprises from 0 to 5%, and preferably, from 0.01 to 4% by weight 12-hydroxystearic acid (12-HSA) and from 0.01 to 25% by weight glycerol. In still another embodiment, the end use composition of the present invention comprises from 0 to 6% by weight sulfate-based surfactant (e.g., sodium lauryl sulfate), but preferably no (0.0% by weight) sulfate-based surfactant.

The viscosity of the end use composition of this invention is typically from 1,000 to 50,000 cps. The wash off composition preferably has a viscosity under 9,000 cps and the leave-on compositions preferably have a viscosity at 6,000 to 15,000 cps. Viscosity may be measured with art recognized instrumentation such as a Brookfield Viscometer RVT, Model 0220, using a T-bar spindle D at 5 RPM, 60 seconds at 25° C.

Illustrative end use compositions suitable to include 0.001 to 6% by weight first component and 0.001 to 6% by weight second component are described below.

Body Lotion for Shower

| Ingredient | Weight % |
|---|---|
| Water | Balance |
| Hydroxypropyl Starch Phosphate | 2.0 |
| Cetyl Phosphate | 1.0 |
| Potassium Hydroxide | 0.5 |
| Hydroxypropyl Bis-Hydroxyethyl Dimonium Chloride | 3.0 |
| Stearamidopropyl PG-Dimonium Chloride Phosphate | 2.0 |
| Shea Butter | 5.0 |
| Coconut Oil | 5.5 |
| Cococaprylate | 2.0 |
| Cetearyl Alcohol | 1.5 |
| Fragrance | 0.1 |

Body Lotion

| Water | Balance |
|---|---|
| Cetyl Hydroxyethylcelllose | 0.25 |
| Sorbitan Oleate Decylglucoside Crosspolymer | 3.5 |
| Potassium Cetyl Phosphate | 1.0 |
| Glycerin | 2.0 |
| Ethylhexyl Palmitate | 3.0 |
| Isopropyl Palmitate | 6.0 |
| Avocado Oil | 3.0 |
| Cetyl Alcohol | 4.0 |
| Diheptyl Succinate and Capryloyl Glycerin/Sebacic | 2.0 |
| Acid Copolymer | |
| Fragrance | 0.1 |

Shampoo Composition

| Water | Balance |
|---|---|
| Cocamidopropyl Betaine | 20.0 |
| Sodium Bis-Hydroxyethylglycinate Coco-Glucoside | 6.0 |
| Crosspolymer | |
| Sodium Methyl Cocoyl Taurate | 2.0 |
| Sodium Stearoyl Lactylate | 10.0 |
| Fragrance | 0.06 |

When preparing such compositions, stirring is used at atmospheric pressure with moderate heat (50 to 60° C.) until homogeneous compositions are obtained. Homogenization is preferably included.

A wide variety of packaging can be employed to store and deliver the end use composition comprising the antibacterial system of this invention. Packaging is often dependent upon the type of product and its use. For instance, leave-on skin lotions and creams, shampoos, conditioners and shower gels generally employ plastic containers with an opening at a dispensing end covered by a closure. Metallic can pressurized by a propellant and having a spray nozzle serve as packaging for mousses and other personal care products.

The following example is provided to facilitate an understanding of the present invention. The example is not intended to limit the scope of the claims.

### Example

The surprising synergistic benefits obtained using the natural antibacterial ingredients of the present invention were confirmed by performing studies similar to the one described below.

Naturally derived antibacterial systems were made, as shown in Table II, by combining thymol with one of p-anisic acid, aloe, gluconolactone, tetrahydrocurcumin and 4-hydroxyacetophenone. Two pools were set up and each pool contained 2 replications. The strains involved were Pseudomonas aeruginosa, Pseudomonas putida, Burkholderia cepacia, Enterobacter gergoviae and Klebsiella pneumoniae. FIC test range (%) was 2.0-0.008 for all samples. The first and second natural components (Components A and B) were added to make a 50/50 mixture of natural component antibacterial system.

The differing behaviors of inhibitory antibacterials in isolation and mixtures have been widely explored using the concept of the Fractional Concentration and Fractional Inhibitory Concentration (FIC). See for instance JRW Lambert and R Lambert, J. Appl. Microbiol 95, 734 (2003); T. Jadavji, CG Prober and R Cheung, Antibacterial Agents and Chemotherapy 26, 91 (1984), and WO 2004/006876. MIC is the minimum inhibitory concentration a component prevents visible growth of a bacterium or bacteria. The FC and FIC parameters can be defined as follows:
FC (Component A)=Concentration of Component A tested in the mixture/MIC (Component A tested as a single active).
FIC (component a)=MIC (component a tested in the mixture)/MIC (component a tested as a single active).

The interactions between antibacterials can be additive, synergistic or possibly antagonistic depending on whether the efficacy of the combination is equivalent to, greater than or less than that obtained for the same total concentration of the individual components when tested alone.

These relationships can be expressed mathematically by summing the fractional MIC values for all the components present in the mixture to give the “fractional inhibitory index”:

ΣFIC=FIC_{(component 1)}+FIC_{(component 2)}

Such that:
ΣFIC greater than 0.5 corresponds to indifferent, additive or antagonistic activity
ΣFIC less than or equal to 0.5 corresponds to synergistic activity

A comparable method is the calculation of the synergy index (SI) which is an industrial accepted method described by Kull, F. C.; Eisman, P. C.; Sylwestrowicz, H. D. and Mayer, R. L., in Applied Microbiology 9:538-541 (1961).

Test Method

Liquid broth assays (MIC and checkerboard) were conducted to identify the minimum concentration(s) of individual and binary combinations of preservation chemicals. A modified methodology to ISO 20776-1:2006 was utilized for the screening as follows. Stock solutions of preservation chemicals and tryptic soy broth were inoculated with 1-5×10⁶ microorganisms and incubated at 30° C. for 24 hours, after which optical densities at OD₆₀₀ nm were measured. MIC was defined as the concentration at which <25% growth was observed in comparison to a positive growth control containing no preservation chemicals. Preservation chemicals were screened at a concentration range of 0.008-2%.

TABLE I
Microorganism pools

| | |
|---|---|
| Pool 1 | Gram negative non-fermenting bacteria: Pseudomonas |
| | aeruginosa, Pseudomonas putida & Burkholderia |
| | cepacia |
| Pool 2 | Gram negative fermenting bacteria: Enterobacter gergoviae & |
| | Klebsiella species |

TABLE II
Synergistic combinations

| | | Pool 1 | | | | Pool 2 | | | |
|---|---|---|---|---|---|---|---|---|---|
| Comp- | Comp- | Replicate 1 | | Replicate 2 | | Replicate 1 | | Replicate 2 | |
| onent | onent | FIC | Inter- | FIC | Inter- | FIC | Inter- | FIC | Inter- |
| A | B | Index | action | Index | action | Index | action | Index | action |
| Thymol | p-Anisic | 0.3 | Syner- | 0.3 | Syner- | 0.3 | Syner- | 0.1 | Syner- |
| | Acid | | gism | | gism | | gism | | gism |
| Thymol | Leucidal | 0.3 | Syner- | 0.3 | Syner- | 0.2 | Syner- | 0.2 | Syner- |
| | Advanced | | gism | | gism | | gism | | gism |
| | Aloe* | | | | | | | | |
| Thymol | Gluconola- | 0.3 | Syner- | 0.1 | Syner- | 0.5 | Syner- | 0.3 | Syner- |
| | ctone | | gism | | gism | | gism | | gism |
| Thymol | Tetrahydro- | 0.1 | Syner- | 0.1 | Syner- | 0.2 | Syner- | 0.4 | Syner- |
| | curcumin | | gism | | gism | | gism | | gism |
| Thymol | 4-Hydroxy- | 0.3 | Syner- | 0.2 | Syner- | 0.5 | Syner- | 0.6 | Indiff- |
| | acetophen- | | gism | | gism | | gism | | erence |
| | one | | | | | | | | |

*Supplied as Water & Leuconostoc/Sorbus aucuparia Fruit Ferment Filtrate

The data provided in Table II surprisingly shows that when combining natural antibacterial ingredients consistent with the present invention, a synergistic benefit against gram negative non-fermenting and fermenting bacteria is observed.

1-8. (canceled)

9. An antibacterial system consisting essentially of:
a) a first component which is thymol; and (b) a second component which is p-anisic add, aloe, gluconolactone, tetrahydrocurcumin, 4-hydroxyacetophenone or a mixture thereof wherein the first and second component are at a weight ratio 1:6 to 6:1.

10. The antibacterial system according to claim 9 wherein the system consists of the first and second component.

11. An end use composition comprising the antibacterial system according to claim 9 wherein the end use composition comprises from 40 to 75% by weight water and further wherein the end use composition comprises no more than 2% by weight of preservative in addition to the antibacterial system.

12. The end use composition to claim 11 wherein the end use composition comprises no more than 1% by weight of preservative in addition to the antibacterial system.

13. The end use composition according to claim 11 wherein the end use composition comprises no more than 1% by weight of preservative in addition to the antibacterial system.

14. The end use composition according to claim 11 wherein the end use composition comprises no more than 0.6% by weight of preservative in addition to the antibacterial system.

15. The end use composition according to claim 11 wherein the end use composition comprises no preservative in addition to the antibacterial system.

16. The end use composition according to claim 11 wherein the end use composition comprises 0,001 to 6% by weight of the first component and 0.001 to 6% by weight of the second component.

17. The end use composition according to claim 11 wherein the end use composition is a body wash, shampoo, or topical skin composition.

18. The end use composition according to claim 11 wherein the end use composition is a cream, lotion, serum, gel, balm, deodorant, antiperspirant, oral care composition, conditioner, wash bar, liquid wash product or a home care composition.

19. The end use composition according to claim 11 further comprising salicylic acid, niacinamide, 12-hydroxystearic acid, retinol, resorcinol or a mixture thereof.

20. The end use composition according to claim 11 further comprising ceramide, sunscreen, glycerol or a mixture thereof.

21. The end use composition according to claim 11 wherein the second component is gluconolactone, tetrahydrocurcumin, 4-hydroxyacetophenone or a mixture thereof.

22. The end use composition according to claim 11 wherein the second component is 4-hydroxyacetophenone.

23. The end use composition according to claim 11 wherein the second component is tetrahydrocurcumin.

24. The end use composition according to claim 11 wherein the second component is gluconolactone.

25. The end use composition according to claim 11 wherein the end use composition further comprises an isethionate, betaine, taurate, polyglucoside or a mixture thereof.

26. The end use composition according to claim 11 wherein the end use composition further comprises retinal palmitate, retinal propionate, vitamin 82, vitamin E, folic acid, biotin, ascorbyl tetraisopalmitate, magnesium ascorbyl phosphate, ascorbyl glycoside, tocopheryl acetate, tocopheryl palmitate, tocopheryl linoleate, 4-hexyl resorcinol, 4-ethyl resorcinol, petroselinic acid, conjugated linoleic acid, beta hydroxy acid, or a mixture thereof.

27. The end use composition according to claim 11 wherein the end use composition further comprises 12-hydroxystearic acid, 4-hexyl resorcinol, 4-ethyl resorcinol, niacinamide or a mixture thereof.
